(19) United States
(12) Patent Application Publication
(10) Pub. No.: US 2003/0066527 A1
(43) Pub. Date: Apr. 10, 2003

(54) Face mask having device for drawing air into the mask

(76) Inventor: Yen Ling Chen, Taichung (TW)

Correspondence Address: Yen Ling Chen, P.O. Box 63-99, Taichung, 406, TW

(21) Appl. No.: 09/972,653

(22) Filed: Oct. 9, 2001

(51) Int. Cl. A62B 9/04; A62B 7/00; A61M 16/00; A62B 18/02

(52) U.S. Cl. 128/204.18; 128/205.25; 128/202.27; 128/912

(57) ABSTRACT

A face mask includes a fan device for drawing air into the mask and for facilitating the breathing of the users. A holder is secured to the mask and has a chamber for receiving the fan device. A motor may be used for driving the fan device. A canister is disposed in front of the fan device for filtering air. The holder includes a conduit having a mouth engaged into the mask. A check valve is disposed in the mouth for preventing air from flowing backward from the mask to the conduit. A guard member is disposed in front of the fan device for protecting the fan device.

### BACKGROUND OF THE INVENTION

1. Field of the Invention

The present invention relates to a face mask, and more particularly to a face mask having a device for drawing air into the mask.

2. Description of the Prior Art

Typical face masks may be engaged onto the face or the nose portion of the users, and include a filter device provided therein for filtering the air. Due to the provision of the filter device, the air may not be easily breathed into the face mask.

U.S. Pat. No. 5,243,972 to Huang discloses another typical face mask including an oxygen tank coupled to the face mask for supplying the oxygen into the face mask and for facilitating the breathing of the users. However, the oxygen may be consumed, and the users also may not breathe well after the oxygen has been consumed.

The present invention has arisen to mitigate and/or obviate the afore-described disadvantages of the conventional face masks.

### SUMMARY OF THE INVENTION

The primary objective of the present invention is to provide a face mask including a device for drawing air into the mask and for facilitating the breathing of the users.

In accordance with one aspect of the invention, there is provided a face mask comprising a mask body, and means for drawing air into the mask body.

The drawing means includes a fan device for drawing the air into the mask body. The drawing means further includes a holder secured to the mask body, the holder includes a chamber formed therein for receiving the fan device.

A canister is further provided and disposed in front of the fan device for filtering air. The holder includes a housing for receiving the canister, and a cover engaged onto the housing for retaining the canister in the housing.

The mask body includes an aperture formed therein, the holder includes a conduit extended therefrom and having a mouth engaged into the aperture of the mask body, the conduit is communicating with the chamber of the holder for receiving air drawn by the fan device.

A check valve is further provided and disposed in the mouth of the conduit for preventing air from flowing backward from the mask body to the conduit.

A device is further provided for securing the mouth of the conduit to the mask body and includes at least one lock bulge extended radially outward from the mouth of the conduit, and a lock ring engaged onto the mouth and having at least one notch formed therein for receiving the lock bulge and for detachably locking the mouth of the conduit to the mask body.

The holder includes a casing extended therefrom, the drawing means includes a motor disposed in the casing and coupled to the fan device for driving the fan device. The mask body includes an orifice formed therein for receiving the casing, and means for securing the casing to the mask body.

A guard member is further provided and disposed in front of the fan device for protecting the fan device. The guard member includes a passageway formed therein for air circulation purposes, and a net disposed in the passageway for preventing objects from entering into the fan device.

Further objectives and advantages of the present invention will become apparent from a careful reading of a detailed description provided hereinbelow, with appropriate reference to accompanying drawings.

### BRIEF DESCRIPTION OF THE DRAWINGS

FIG. 1 is a plan view of a face mask in accordance with the present invention;

FIG. 2 is a plan and exploded view of the face mask;

FIG. 3 is a partial cross sectional view of the face mask;

FIG. 4 is a partial exploded view of the face mask; and

FIG. 5 is another partial exploded view of the face mask.

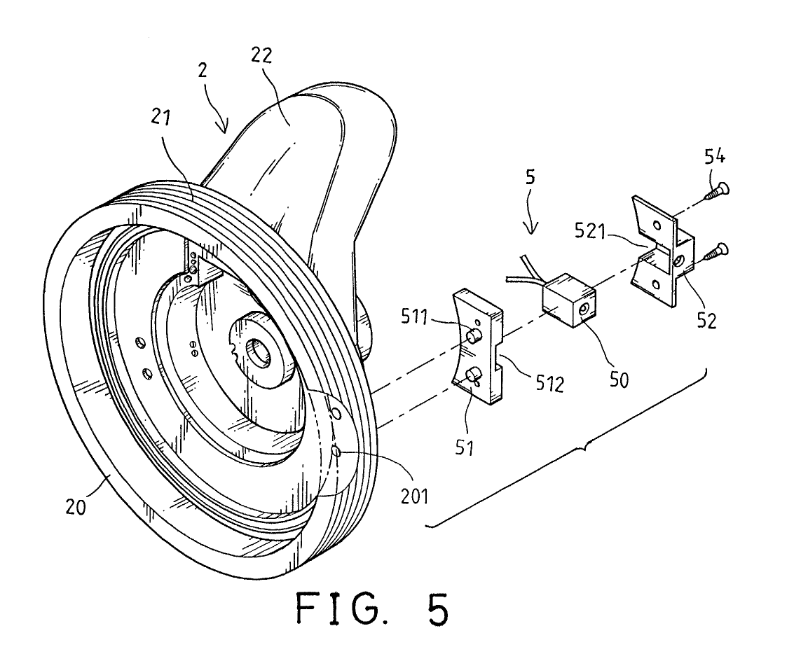

### DETAILED DESCRIPTION OF THE PREFERRED EMBODIMENT

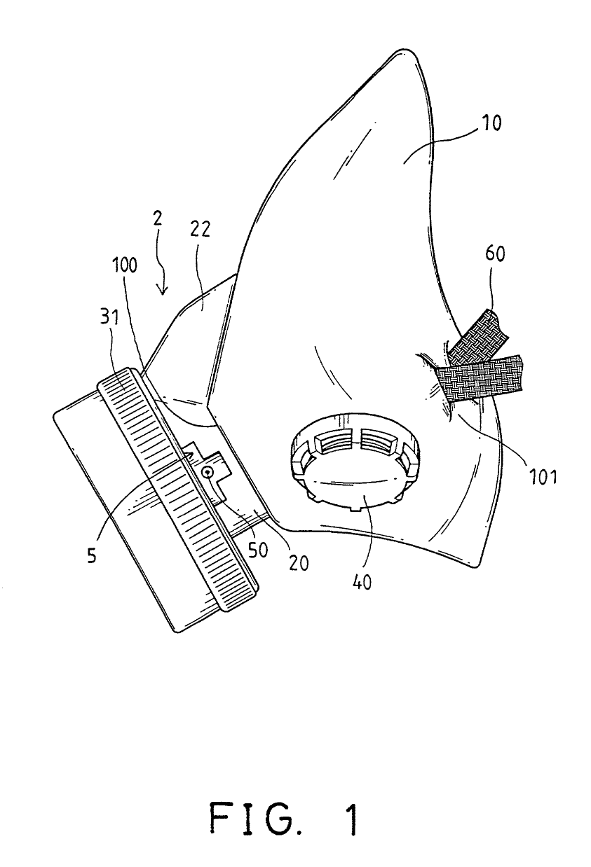

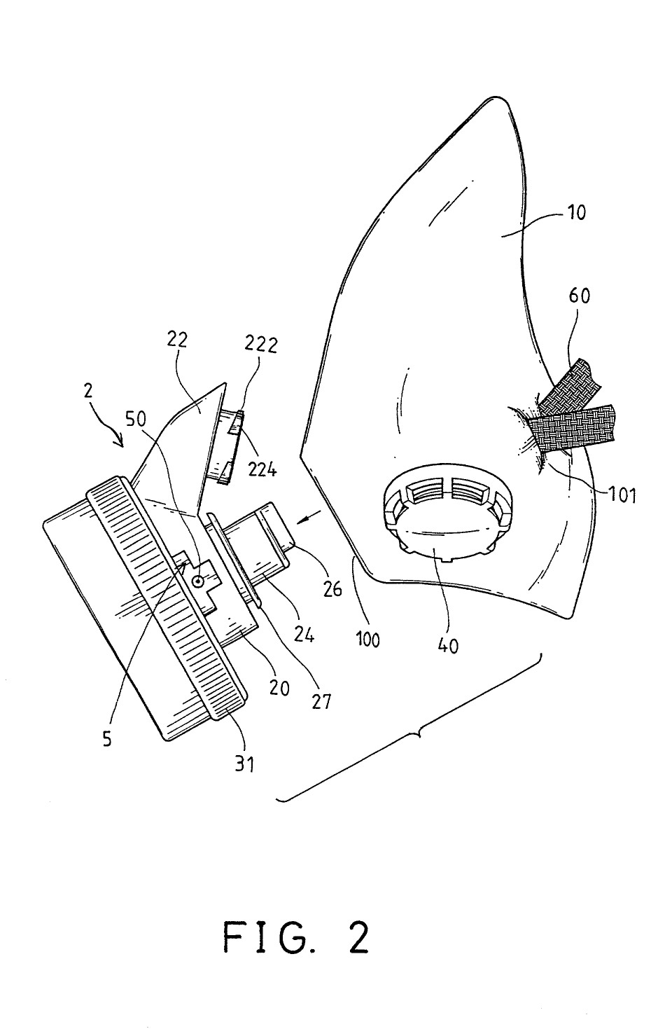

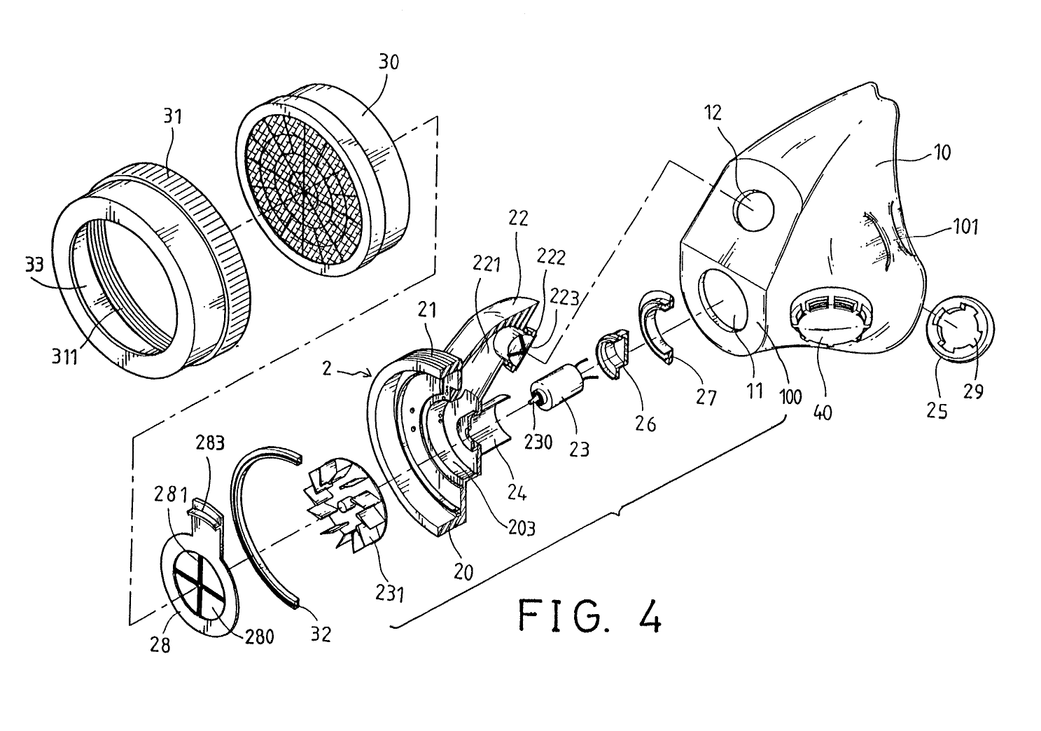

Referring to the drawings, and initially to FIGS. 1-4, a face mask in accordance with the present invention comprises a mask body 10 including a plane surface 100 formed in the front and lower portion thereof and slightly inclined or facing downward, best shown in FIGS. 1-3, and including an orifice 11 formed in the plane surface 100, and including an aperture 12 formed in the front and upper portion thereof, and including one or more outlet ports 40 formed or provided in the bottom portion thereof for allowing the air to flow out of the mask body 10. The mask body 10 includes one or more hooks or engaging members 101 provided thereon for engaging with the fastening strap 60 which may attach or fasten the mask body 10 onto the face or the nose portion of the users.

A holder 2 includes a housing 20, such as a cylindrical housing 20 formed or provided therein for receiving a canister 30 or the like therein. The canister 30 may be any kind of suitable filter devices or the like. The housing 20 includes an outer thread 21 formed on the outer peripheral portion thereof. A cover 31 includes an inner thread 311 formed or provided therein for engaging with the outer thread 21 of the housing 20 and for retaining the canister 30 within the housing 20, and includes an opening 33 formed therein, for allowing the air to flow into the cover 31 and to flow through the canister 30 and for allowing the air to be filtered by the canister 30. A gasket or a sealing ring 32 is further provided and engaged between the canister 30 and the housing 20 for making an air tight seal between the canister 30 and the housing 20.

A protective device or a guard member 28 is further provided and disposed between the canister 30 and the fan device 231, and includes a passageway 280 formed and provided therein for allowing the air to be drawn through the canister 30 by the fan device 231, and for allowing the air to flow into the holder 2. A cross-shaped bar or a net 281 is provided and secured in the passageway 280 of the guard member 28 and disposed in front of the fan device 231 for preventing large objects to be drawn through the fan device 231 and for protecting the fan device 231 and for preventing the fan device 231 from being damaged by the other or larger objects. The guard member 28 preferably includes an extension 283 extended therefrom and engaged into the housing 20 for solidly securing the guard member 28 in the housing 20 or between the housing 20 and the canister 30.

The holder 2 includes a chamber 203 formed or provided therein and communicating with the housing 20 for receiving a fan device 231 therein, and includes a casing 24 formed or provided therein and communicating with the chamber 203 thereof for receiving a motor 23 therein. The casing 24 is engaged into the mask body 10 via the orifice 11 of the mask body 10. The motor 23 includes a spindle 230 engaged into the chamber 203 of the holder 2 and secured to the fan device 231 for driving the fan device 231. A cap 26 is engaged onto the casing 24 for securing and retaining the motor 23 within the casing 24. A gasket or a sealing ring 27 is further provided and engaged between the mask body 10 and the casing 24 for making an air tight seal between the mask body 10 and the casing 24, and for attaching or securing the casing 24 and thus the holder 2 onto the front portion of the mask body 1.

The holder 2 includes a conduit 22 provided thereon or extended therefrom and having a passage 221 formed or provided therein and communicating with the chamber 203 thereof. The conduit 22 includes a mouth 222 extended therefrom and engaged into the aperture 12 of the mask body 10, for allowing the fan device 231 to draw air into the mask body 10 via the passage 221 of the conduit 22. A membrane or a check valve 223 is disposed or provided in the mouth 222 for preventing the air from flow backward from the mask body 10 into the holder 2. The mouth 222 includes one or more lock bulges 224 extended radially outward therefrom. A lock ring 25 is engaged onto the mouth 222 and includes one or more notches 29 formed therein for receiving the lock bulges 224 and for detachably locking the mouth 222 of the conduit 22 to the mask body 10.

A receptacle 5 is further provided and includes a base 51 having one or more pins or fasteners 511 extended therefrom or attached thereto for engaging through the holes 201 of the housing 20 and for securing to the housing 20. The base 51 includes a cavity 512 formed therein for receiving one or more batteries 50 therein, or for receiving the converter or the other electric members therein. A lid 52 is secured to the base 51 with fasteners 54 or the like, and includes a space 521 formed and provided therein for partially receiving the batteries 50 and/or the other electric members therein. The batteries 50 and/or the other electric members may be coupled to the motor 23 for controlling or for actuating or energizing the motor 23 to drive the fan device 231. The motor 23 may also be coupled to the other electric power source for being energized to drive and to actuate the fan device 231.

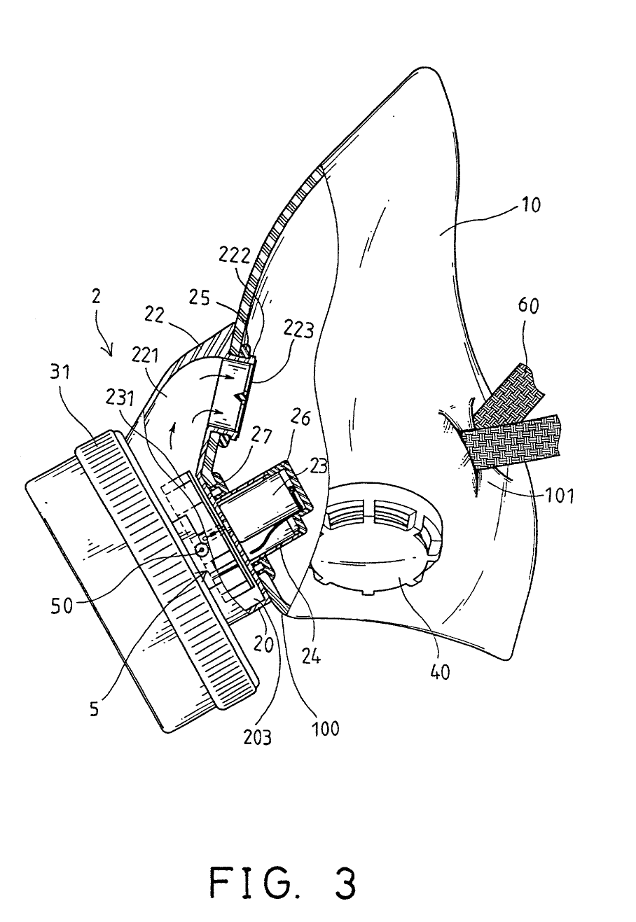

In operation, as shown in FIG. 3, the motor 23 may actuate the fan device 231 to draw the air through the canister 30 and through the passage 221 of the conduit 22 and then into the mask body 10, in order to facilitate the breathing of the users. The check valve 223 may be used for preventing the air from flow backward from the mask body 10 into the holder 2. The holder 2 may be detachably secured onto the mask body 10 with the sealing ring 27 and/or the lock ring 25. The housing 20 of the holder 2 is attached to the inclined surface 100 which is slightly inclined or facing downward for preventing the holder 2 from obstructing the viewing range of the users.

Accordingly, the face mask in accordance with the present invention includes a device for drawing air into the mask and for facilitating the breathing of the users.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. A face mask comprising:
a mask body, and
a fan device attached to said mask body for drawing air into said mask body.

2. A face mask comprising:
a mask body, and
a motor-driven fan device attached to said mask body for drawing air into said mask body.

3. A face mask comprising:
a mask body, and
means for drawing air into said mask body.

4. The face mask according to claim 3, wherein said drawing means includes a fan device for drawing the air into said mask body.

5. The face mask according to claim 4, wherein said drawing means includes a holder secured to said mask body, said holder includes a chamber formed therein for receiving said fan device.

6. The face mask according to claim 5 further comprising a canister disposed in front of said fan device for filtering air.

7. The face mask according to claim 6, wherein said holder includes a housing for receiving said canister, and a cover engaged onto said housing for retaining said canister in said housing.

8. The face mask according to claim 5, wherein said mask body includes an aperture formed therein, said holder includes a conduit extended therefrom and having a mouth engaged into said aperture of said mask body, said conduit is communicating with said chamber of said holder for receiving air drawn by said fan device.

9. The face mask according to claim 8 further comprising a check valve disposed in said mouth of said conduit for preventing air from flowing backward from said mask body to said conduit.

10. The face mask according to claim 8 further comprising means for securing said mouth of said conduit to said mask body.

11. The face mask according to claim 10, wherein said securing means includes at least one lock bulge extended radially outward from said mouth of said conduit, and a lock ring engaged onto said mouth and having at least one notch formed therein for receiving said at least one lock bulge and for detachably locking said mouth of said conduit to said mask body.

12. The face mask according to claim 5, wherein said holder includes a casing extended therefrom, said drawing means includes a motor disposed in said casing and coupled to said fan device for driving said fan device.

13. The face mask according to claim 12, wherein said mask body includes an orifice formed therein for receiving said casing, and means for securing said casing to said mask body.

14. The face mask according to claim 4 further comprising a guard member disposed in front of said fan device for protecting said fan device.

15. The face mask according to claim 14, wherein said guard member includes a passageway formed therein for air circulation purposes, and a net disposed in said passageway for preventing objects from entering into said fan device.
